(19) United States
(12) Patent Application Publication
(10) Pub. No.: US 2023/0162844 A1
(43) Pub. Date: May 25, 2023

(54) PATIENT PROVIDER MATCHING SYSTEM

(71) Applicant: Evernorth Strategic Development, Inc., St. Louis, MO (US)

(72) Inventors: Ching Lau, Arcadia, CA (US); Deepan Vishal Thulasi Vel, Hartford, CT (US); Jinshu Cui, Arcadia, CA (US)

(21) Appl. No.: 17/533,993

(22) Filed: Nov. 23, 2021

(51) Int. Cl. G16H 40/20; G16H 50/20; G06Q 10/06; G06F 16/2457; G06F 16/242; G06F 40/40; G06K 9/62

(52) U.S. Cl. CPC G16H 40/20; G16H 50/20; G06Q 10/063112; G06F 16/24578; G06F 16/2428; G06F 40/40; G06K 9/6272

(57) ABSTRACT

Finding a medical care provider that serves a patient's unique needs is a highly personalized process. Embodiments herein describe a patient provider matching system for providing a personalized experience that highlights factors that are most important for patients in choosing their medical care provider. The patient provider matching system receives a query by a patient user and identifies a list of medical providers that match the query based on provider qualifications and medical claims data. The patient provider matching system, ranks the identified list of medical providers based on patient data and provider data. The patient provider matching system, displays the ranked list of medical providers on a graphical user interface for the patient user. Further details of the patient provider matching system are provided below.

### TECHNICAL FIELD

Systems and methods herein generally relate to generating optimized search results. More specifically, but not by way of limitations, embodiments herein describe a patient provider matching system.

### BACKGROUND

Many patients rely on family and friends for referrals of medical care providers. Other rely on their insurance provider directory or hospital websites.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 is a block diagram of an example implementation of a healthcare system including a patient provider matching system, according to example embodiments.

FIG. 2 illustrates a database associated with the patient provider matching system, according to example embodiments.

FIG. 3 is a block diagram of a patient provider matching system, according to example embodiments.

FIG. 4 is an illustration of a patient provider matching system, according to example embodiments.

FIG. 5 is a flowchart of an example method for generating a ranked list of search results using a patient provider matching system, according to example embodiments

FIG. 6 is a block diagram illustrating a software architecture, which can be installed on any one or more of the devices described herein

FIG. 7 is a diagrammatic representation of the machine within which instructions (e.g., software, a program, an application, an applet, an app, or other executable code) for causing the machine to perform any one or more of the methodologies discussed herein may be executed.

### DETAILED DESCRIPTION

Finding a medical care provider that serves a patient's unique needs is a highly personalized process. Embodiments herein describe a patient provider matching system for providing a personalized experience that highlights factors that are most important for patients in choosing their medical care provider.

The patient provider matching system receives a query by a patient user and identifies a list of medical providers that match the query based on provider qualifications and medical claims data. The patient provider matching system, ranks the identified list of medical providers based on patient data and provider data. The patient provider matching system, displays the ranked list of medical providers on a graphical user interface for the patient user. Further details of the patient provider matching system are provided below.

### Matching System

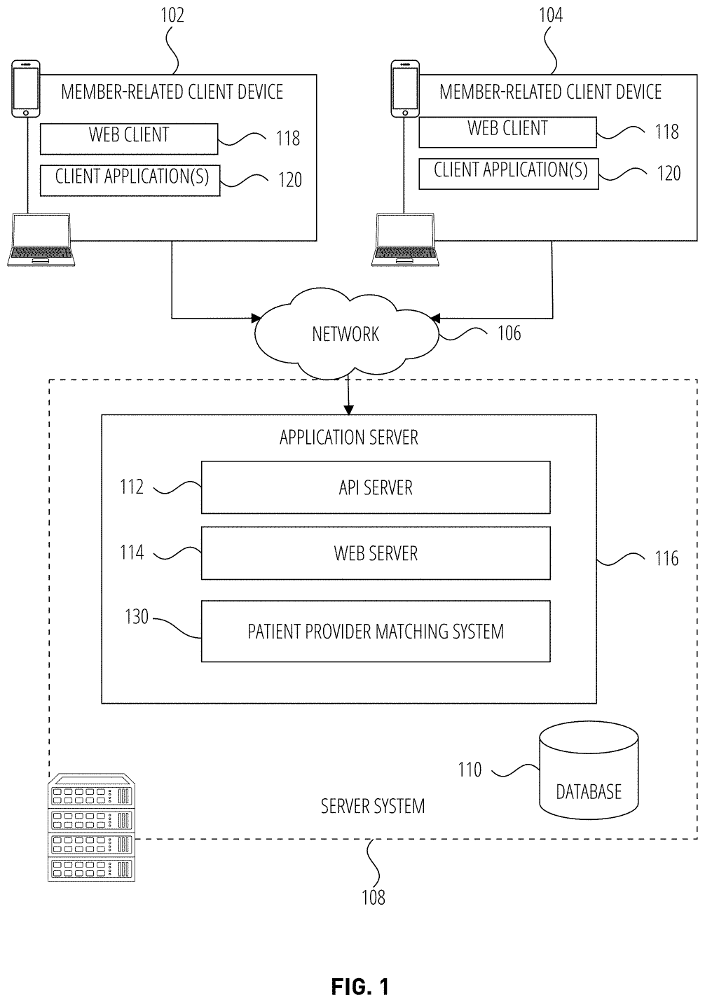

FIG. 1 is a block diagram showing an example healthcare system according to various exemplary embodiments, configured to match patients with providers according to a set of patient preferences. The healthcare system may further be used to check the status of member healthcare claim data, check member healthcare coverage benefits, etc. The healthcare system includes one or more client devices such as member-related client device 102 and member-related client device 104. A client device may be, but is not limited to, a mobile phone, desktop computer, laptop, portable digital assistants (PDAs), smart phones, a wearable device (e.g., a smart watch), tablets, ultrabooks, netbooks, laptops, multi-processor systems, microprocessor-based or programmable consumer electronics, game consoles, set-top boxes, or any other communication device that a user may use to access a network. The member-related client device 102, 104 includes a processor that loads instructions from a memory. With the processor being loaded with specific instructions to perform tasks as described herein sets the user device to a specific purpose computing and communication system. The member-related client device 102 and member-related client device 104 can include a microphone and speaker on a mobile electronic device, a telephone, or a self-service kiosk, e.g., at a pharmacy, a clinic, a doctor's office, a mobile relief center, and the like. The speaker and microphone enable audio communication to and from the member-related client device.

The member-related client device 102 and member-related client device 104 may be devices of users that are used to access and utilize an online healthcare platform. For example, each of the member-related client device 102 and member-related client device 104 may be used to input information to create an account, access a member profile, view patient providers, and so forth. The inputs can be audio or tactile input, e.g., keyboard, touch screen and the like.

For example, member-related client device 102 is a device of a given user who would like to search for or otherwise find a provider for a specific healthcare need. Member-related client device 102 accesses a website of a healthcare platform (e.g., hosted by server system 108). The user inputs login credentials associated with the user. Server system 108 receives the request and provides access to the online healthcare platform.

One or more users may be a person, a machine, or other means of interacting with the member-related client device 102 or member-related client device 104. In example embodiments, the user may not be part of the system but may interact with the system via the member-related client device 102 or member-related client device 104 or other means. For example, a user may provide input to the member-related client device 102 and the input may be communicated to the server system 108 via the network 106.

The healthcare system can further include a network 106. The network 106 may include, or operate in conjunction with, an ad hoc network, an intranet, an extranet, a virtual private network (VPN), a local area network (LAN), a wireless network, a wireless LAN (WLAN), a wide area network (WAN), a wireless WAN (WWAN), a metropolitan area network (MAN), the Internet, a portion of the Internet, a portion of the Public Switched Telephone Network (PSTN), a plain old telephone service (POTS) network, a cellular telephone network, a wireless network, a Wi-Fi® network, another type of network, or a combination of two or more such networks. For example, a network or a portion of a network may include a wireless or cellular network and the coupling may be a Code Division Multiple Access (CDMA) connection, a Global System for Mobile communications (GSM) connection, or other type of cellular or wireless coupling. In this example, the coupling may implement any of a variety of types of data transfer technology, such as Single Carrier Radio Transmission Technology (1×RTT), Evolution-Data Optimized (EVDO) technology, General Packet Radio Service (GPRS) technology, Enhanced Data rates for GSM Evolution (EDGE) technology, third Generation Partnership Project (3GPP) including 3G, fourth generation wireless (4G) networks, fifth generation wireless (5G) networks, Universal Mobile Telecommunications System (UMTS), High Speed Packet Access (HSPA), Worldwide Interoperability for Microwave Access (WiMAX), Long Term Evolution (LTE) standard, others defined by various standard setting organizations, other long range protocols, or other data transfer technology.

The member-related client device 102 and member-related client device 104 may access the various data and applications provided by other entities via the web client 118 (e.g., a browser) or one or more client application(s) 120. The member-related client device 102 and member-related client device 104 may include one or more client application(s) 120 such as, but not limited to, a web browser, a healthcare application, electronic mail (email) application, an e-commerce site application, a mapping or location application, and the like. These functions can be specific to the use of predictive models to generate provider recommendations to user device, e.g., the member-related client device 102, 104.

In some embodiments, one or more client application(s) 120 are included in a given member-related client device 102 and member-related client device 104, and configured to locally provide the user interface at least some of the functionalities, with the client application(s) 120 configured to communication with the server system 108 on an as-needed bases, for data processing capabilities not locally available. Conversely, one or more client application(s) 120 may not be included in the member-related client device 102 or member-related client device 104, and the client devices may use its web browser to access the one or more applications hosted on the server system 108.

A server system 108 provides server-side functionality via the network 106 to the member-related client device 102 and member-related client device 104. The server system 108 includes an application program interface (API) Server 112, a Web Server 114 and patient provider matching system 130, that may be communicatively coupled with one or more database(s) 110. The one or more database(s) 110 may be storage devices that store data related to users of the server system 108, applications associated with the server system 108, cloud services, and so forth. In one example, the one or more database(s) 110 may be cloud-based storage.

The server system 108 may be cloud computing environment, according to some example embodiments. The server system 108, and any servers associated with the server system 108 may be associated with a cloud-based application, in one embodiment.

The server system 108 includes a patient provider matching system 130. The patient provider matching system 130 enables a patient user using a patient device (e.g. member-related client device 102 and member-related client device 104) to search for providers that match their preferences. The patient provider matching system 130 generates a ranked list of providers based on medical claim data and the patient user's preferences. The ranked list generated by the patient provider matching system 130 further provides additional data about each provider that a patient user may find beneficial when selecting a provider.

In some examples, the healthcare system includes a customer service client device (not pictured). The customer service client device may be used to establish a communication session (e.g., via a network 106) to one or more of the member-related client device 102 and member-related client device 104. The customer service client device may further be used to access and utilize functionalities of the server system 108.

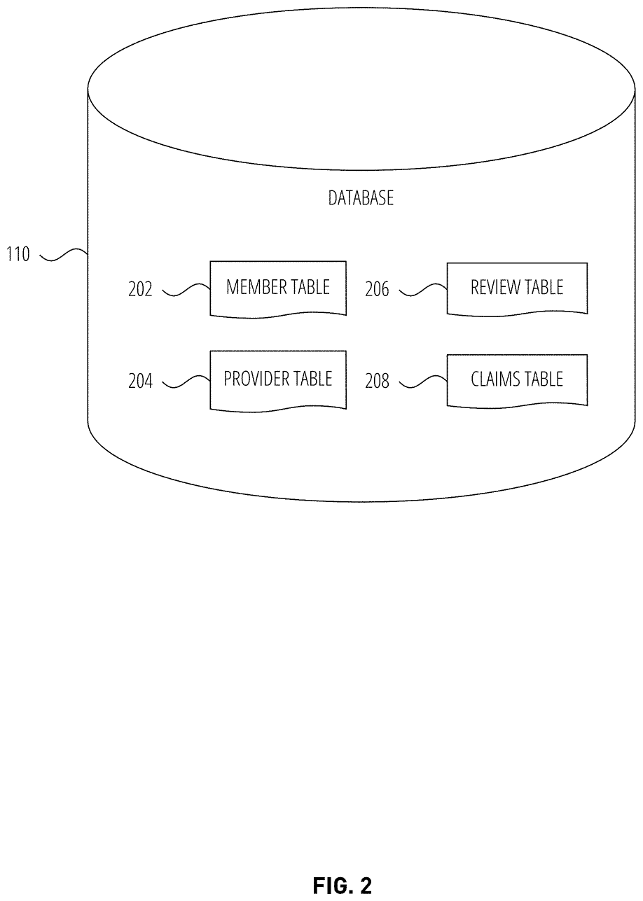

FIG. 2 is a schematic diagram illustrating data that is stored in the database 110 of the server system 108, according to certain exemplary embodiments. While the content of the database 110 is shown to comprise a number of tables, the data could be stored in other types of data structures (e.g., as an object-oriented database). The database 110 is shown to include a member table 202, a provider table 204, review table 206, and claims table 208.

The member table 202 includes information regarding the members associated with the healthcare system. The information stored as member table 202 may include personal information, personal health information, protected health information, etc. Examples of the member table 202 include name, address, telephone number, e-mail address, prescription drug history, etc. The member table 202 may include a plan sponsor identifier that identifies the plan sponsor associated with the member and/or a member identifier that identifies the member to the plan sponsor. The member table 202 may include a member identifier that identifies the plan sponsor associated with the user and/or a user identifier that identifies the user to the plan sponsor. The member table 202 may also include dispensation preferences such as type of label, type of cap, message preferences, language preferences, etc.

The member table 202 may be accessed by the patient provider matching system 130 for review, verification, or other purposes. In general, the use of the terms “member” and “user” may be used interchangeably.

The provider table 204 includes information regarding the providers associated with the healthcare system. Examples of the provider table 204 include name, address, telephone number, e-mail address, speciality, education background, practice history, etc. The provider table 204 may further include provider ratings, and indicators that represent whether a specific provider is a recommended provider.

The review table 206 includes information regarding member reviews of a given provider. Examples of the review table 206 include phrases, full sentences, partial sentences, or any suitable textual description of a given provider written by a member.

The claims table 208 includes information regarding pharmacy claims. In general, the claims table 208 includes an identification of the client that sponsors the drug benefit program under which the claim is made, and/or the member that purchased the prescription drug giving rise to the claim, the prescription drug that was filled by the pharmacy (e.g., the national drug code number, etc.), the dispensing date, generic indicator, generic product identifier (GPI) number, medication class, the cost of the prescription drug provided under the drug benefit program, the copayment/coinsurance amount, rebate information, and/or member eligibility, etc. Additional information may be included.

In some implementations, other types of claims beyond prescription drug claims may be stored in the claims table 208. For example, medical claims, dental claims, wellness claims, or other types of health-care-related claims for members may be stored as a portion of the claims table 208.

In some implementations, the claims table 208 includes claims that identify the members with whom the claims are associated. Additionally or alternatively, the claims table 208 may include claims that have been de-identified (that is, associated with a unique identifier but not with a particular, identifiable member).

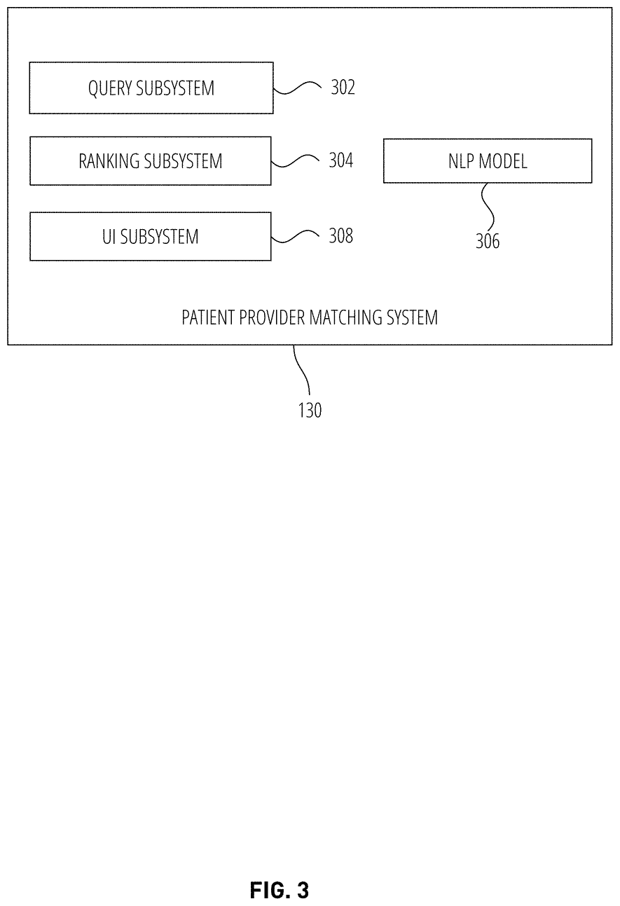

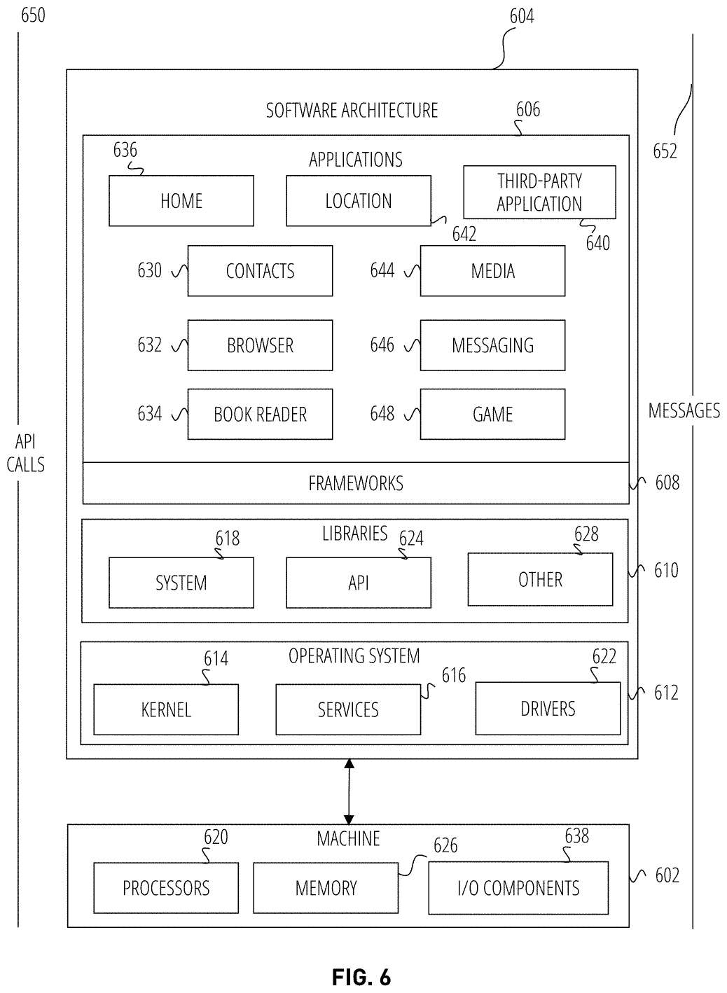

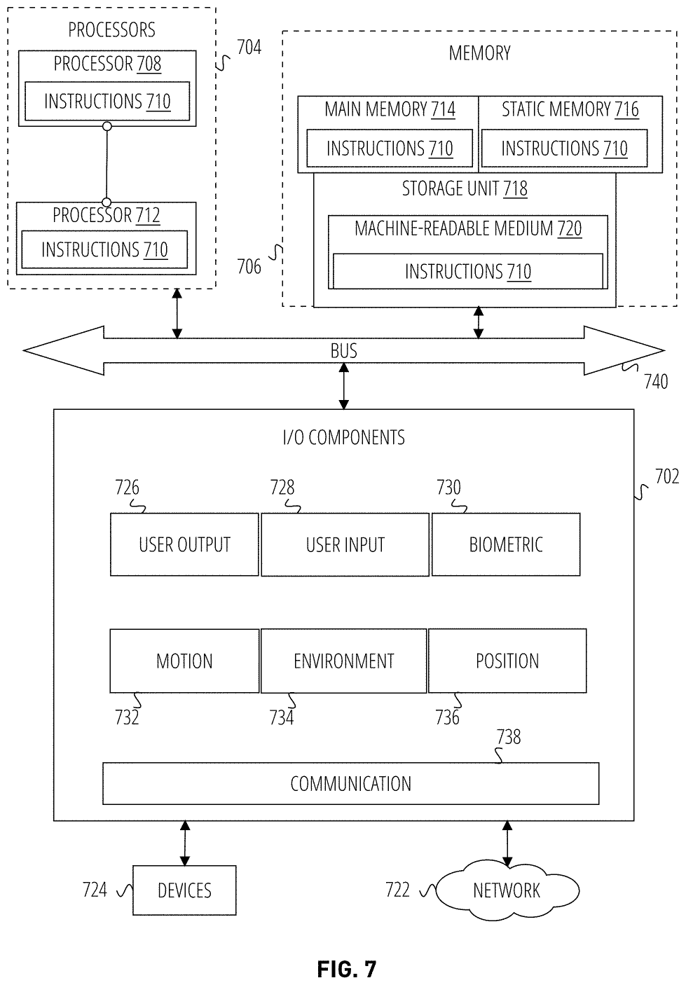

The patient provider matching system 130 in FIG. 3 includes a query subsystem 302, ranking subsystem 304, an NLP Model 306, and a UI Subsystem 308. The patient provider matching system 130 can further include elements described with respect to FIG. 6 and FIG. 7 as a processor and memory, having instructions stored thereon, that when executed by the processor, causes the processor to control the functions of the patient provider matching system 130.

The query subsystem 302 receives a query provided by the patient user. The query includes a set of patient preferences associated with the patient user. The patient user may provide the query as input via a graphical user interface provided by the UI Subsystem 308. For example, the UI Subsystem 308 may display a text field, drop down elements, checkboxes, or any suitable interactive user interface elements for receiving the query. In some examples, the query received by the query subsystem 302 is a search for medical providers for the patient user. The patient preferences include the patient user's requirements for a medical provider that is recommended by the patient provider matching system 130. For example, patient preferences may include a practice area of the provider, age of the patient, location of the patient and/or provider, and the like. The present system may have organized the potential providers using a predictive model of possible providers. Such a predictive model reviews claim history and provider data to generate predictions on best provider fits for groups of members. In an example embodiment, the system generates a list of individuals ordered according to corresponding probability values, wherein the machine learning model determines a probability value for a provider match. The system can use an artificial intelligence engine applying at least one machine learning model configured to provide a probability value for each provider and within groups of providers. The listing or probability can be used to generate listings of providers to a member-related client device. In some embodiments, the at least one machine learning model includes a multi-layer perceptron model. In some embodiments, the machine learning model includes a fully-connected multi-layer perceptron model. In some embodiments, the machine learning model includes at least an input layer, a hidden layer, and an output layer. In some embodiments, the machine learning model is initially trained using a supervised learning technique. In some embodiments, the machine learning model is iteratively trained using at least the output of the at least one machine learning model. In some embodiments, the machine learning model applies a linear regression model.

The ranking subsystem 304 identifies search results (e.g., medical providers) that match the query received by the query subsystem 302 and generates a ranked list of search results. To identify search results that match the query, the ranking subsystem 304 may access the location of the patient user's device (e.g., member-related client device 102, member-related client device 104), data from the database 110, for example.

In some examples, the ranking subsystem 304 uses the centroid of the geographic location of the patient user (or patient user's device) to identify search results that match the query. Based on the centroid of the location of the patient user, the ranking subsystem 304 identifies search results that fall within a predefined radius of the centroid (e.g., 2 miles, 5 miles, 50 miles, etc.).

In some examples, the ranking subsystem 304 uses the centroid of the closest city associated with the patient user (or patient user's device) to identify search results that match the query. Based on the centroid of the closest city, the ranking subsystem 304 identifies search results that fall within a predefined radius of the centroid. If the ranking subsystem 304 identifies a number of search results that does not exceed a minimum threshold amount, the ranking subsystem 304 may extend the radius of the centroid until the ranking subsystem 304 identifies a number of search results that meets or exceeds the minimum threshold amount.

In some examples, the ranking subsystem 304 uses both the centroid of the geographic location of the patient user (or patient user's device) and the centroid of the closest city associated with the patient user (or patient user's device) to identify search results that match the query. If the ranking subsystem 304 determines that the centroid of the geographic location exceeds a threshold distance from the centroid of the closest city, the ranking subsystem 304 may generate a notification or trigger an alert to a member-related client device 102 or member-related client device 104. In some examples, the ranking subsystem 304 may predictively submit a request to schedule an appointment with a preferred provider. A user is prompted to confirm or deny the scheduling request from a user interface. If the user confirms the appointment, the ranking subsystem 304 may request the patient provider matching system 130 to modify the provider's availability. In an example, the ranking system 304 may provide an selectable scheduling icon to the member-related client device. The user may select the icon to launch a process that links the member-related client device to the scheduling system, e.g., a computing system, of the provider selected by the user on their related member-related client device. A communication link is established directly between the member-related client device and provider scheduling system. This communication link bypasses the scheduling system.

In some examples, the ranking subsystem 304 analyzes claims data from the claims table 208 to determine which medical providers that patient users in a given region seek care from. In another example, based on the geographic location of the patient user (or patient user's device), the ranking subsystem 304 may use the claims data to identify neighboring regions that patient users seek medical care from medical providers. The ranking system 304 may use the claims data to generate a predictive model of medical providers. The ranking subsystem 304 may require that the neighboring regions be within a predefined distance from the primary given region that represents the patient's location. For example, if the patient user lives in a city, the ranking subsystem 304 may use the claims data to determine which medical providers other patient users in the city seek medical care from. However, if the patient user lives in a rural area, the ranking subsystem 304 may need to identify neighboring regions in order to provide the patient user with a more exhaustive list of options of medical providers. The ranking subsystem 304 may require that the identified neighboring regions be within a reasonable distance (e.g., within a 30 minute drive) from the patient's location. The ranking subsystem 304 may further require that a specific percentage of residents from the patient's primary location travel to the identified neighboring regions to seek medical care.

In some examples, the ranking subsystem 304 may identify multiple locations for a single provider. In that instance, the ranking subsystem 304 only retains the location closest to the patient and does not include other locations of the provider in the final ranked list.

The ranking subsystem 304 further includes a predictive model that generates a probability that the patient user will select a given identified provider. The predictive model receives the identified list of providers, patient information and provider information as input and generates an output that represents the probability that the patient user will select the given identified provider. In some examples the predictive model is a logistics regression model. It is to be understood that the predictive model may be any suitable machine learning model or statistical model.

Based on the probability generated by the predictive model discussed above, the ranking subsystem 304 generates a ranked list of search results. The UI Subsystem 308 causes display of the ranked list of search results on a graphical user interface. Each search result may be displayed as a selectable user interface element (e.g., a button, drop down menu, etc.) that includes the given provider's image, name, professional background, practice area, patient rating, patient review highlights, and further information. A patient user may interact with the ranked list of search results via the UI Subsystem 308 by clicking, tapping, or otherwise selecting a search result and learning more information about the recommended medical providers.

The patient review highlights discussed above may be generated by the Natural Language Processing (NLP) Model 406. For example, the NLP Model 306 may analyze verified patient reviews for a given provider and generate review highlights that a new patient user may find helpful when deciding which provider to select. For example, the NLP Model 306 extracts aspects and sentiments from verified patient reviews The review highlights may highlight various aspects of the provider including but not limited to aspects of the provider such as whether the provider follows up, has good bedside manner, is knowledgeable, attentive, thorough and professional. The review highlights may also hight aspects of the staff such as whether the staff is friendly and professional. The review highlights may also highlight aspects of the office, such as whether the office is an efficient office, the patient had a pleasant experience, the office is kid friendly, it is easy to make an appointment, and the wait time is short.

In some examples, the NLP Model 306 uses two sub-models. The first sub-model classifies review segments to positive or non-positive sentiments and the second sub-model classifies review segments to negative or non-negative sentiments.

In some examples, the UI Subsystem 308 further includes a visualization of a map indicating the locations of each of the providers provided by the ranking subsystem 304. The UI Subsystem 308 may provide functionality for a patient user to interact with the map.

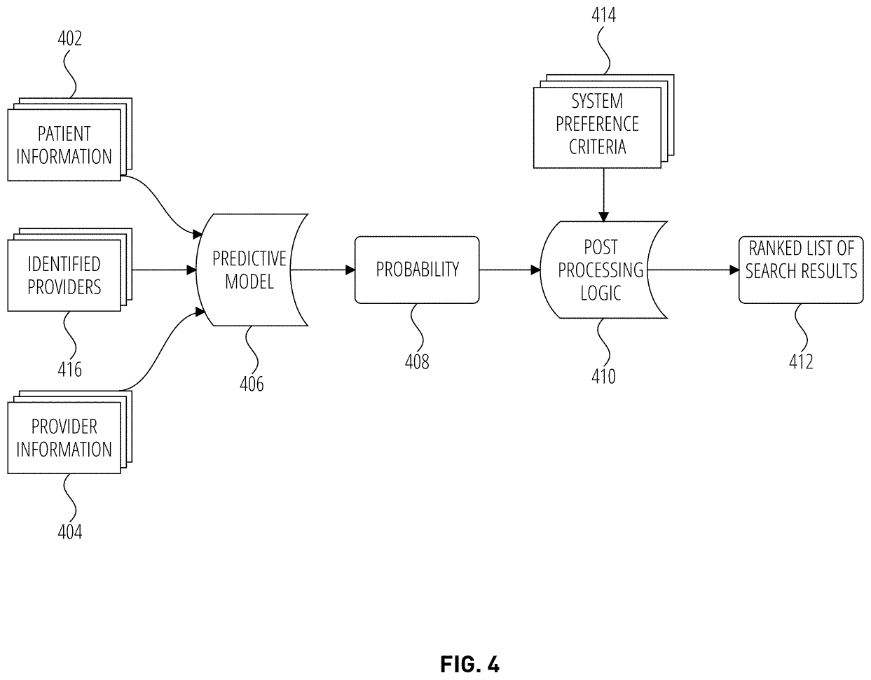

FIG. 4 is an illustration of a patient provider matching system 130, according to example embodiments.

The predictive model 406 receives patient information 402, identified providers 416, and provider information 404 as input. As discussed above in connection with FIG. 3, the ranking subsystem 304 first identifies a list of providers that matches the query received by the query subsystem 302. The patient information 402 includes but is not limited to the patient's age and patient's gender. The patient information 402 may further include other aspects of the member data from the member table 202. The provider information 404 includes but is not limited to the provider's gender, speciality practice areas, distance from the patient, years of experience, patient insights, and review highlights. As discussed above, the review highlights are generated by the NLP Model 306.

The predictive model 406 generates a probability 408 for each provider that the patient user will select the given provider. The patient provider matching system 130 performs post processing logic 410 before providing the final ranked list of search results 412 by analyzing the probability 408 and a set system preference criteria 414. The system preference criteria 414 may include the number of patient reviews for the provider, the overall rating of the provider, cost efficiency of the provider's practice, whether the provider is accepting new patients, and the like. The system preference criteria 414 may be related to preferences set by the healthcare system. Further details of the healthcare system may be found in connection with FIG. 1.

After the post processing logic 410, the patient provider matching system 130 generates a ranked list of search results 412. The ranked list of search results 412 is displayed on a graphical user interface and presented to the patient user who submitted the initial query.

Although the described flowcharts can show operations as a sequential process, many of the operations can be performed in parallel or concurrently. In addition, the order of the operations may be re-arranged. A process is terminated when its operations are completed. A process may correspond to a method, a procedure, an algorithm, etc. The operations of methods may be performed in whole or in part, may be performed in conjunction with some or all of the operations in other methods, and may be performed by any number of different systems, such as the systems described herein, or any portion thereof, such as a processor included in any of the systems.

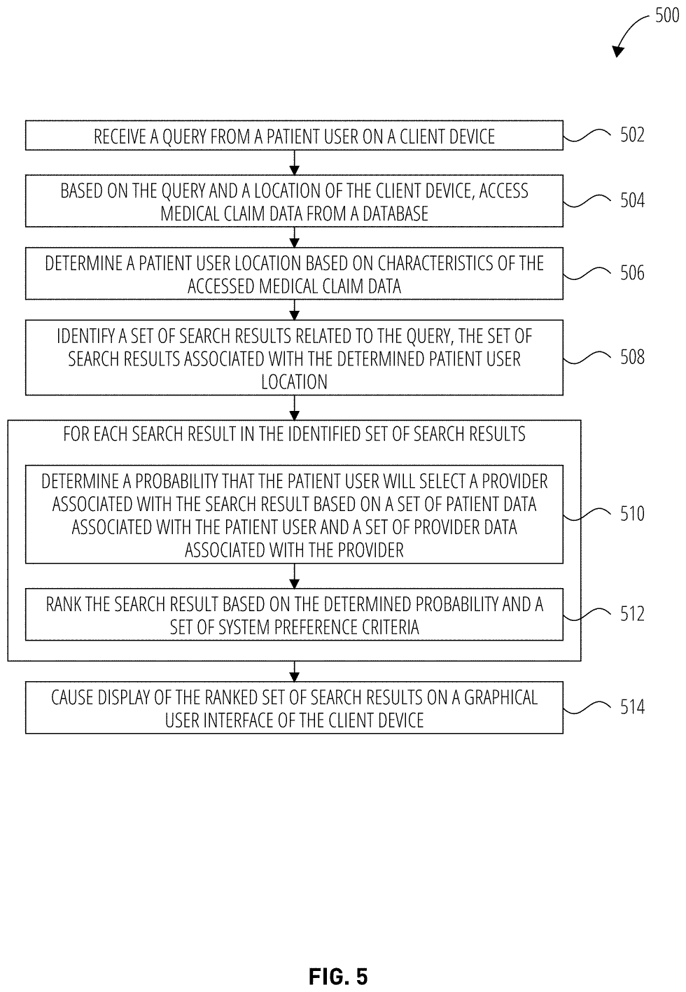

FIG. 5 is a flowchart of an example method 600 for generating a ranked list of search results using a patient provider matching system 130, according to example embodiments. In one example, the processor in a patient provider matching system 130, the processor in member-related client device 102, member-related client device 104, or any combination thereof can perform the operations in the method 600.

At operation 502, the patient provider matching system 130 receives a query from a patient user on a first client device. For example, the query may be received by the query subsystem 302.

At operation 504, based on the query and a location of the client device, the patient provider matching system 130 accesses medical claim data from a database. The medical claim data may be the claims data from the claims table 208. At operation 506, the patient provider matching system 130 determines a patient location based on characteristics of the accessed medical claim data. In some examples, the patient location is based on accessed medical claim data, a centroid of the geographic location of the patient, the centroid of the closest city to the patient, or any combination thereof.

At operation 508, the patient provider matching system 130 identifies a set of search results related to the query. The set of search results are associated with the determined patient user location.

For each search result in the set of search results, the patient provider matching system 130 determines a probability that the patient user will select a provider associated with the search result based on a set of patient data associated with the patient user and a set of provider data associated with the provider at operation 510 and ranks the search result based on the determined probability and a set of system preference criteria at operation 512. For example, the probability is determined using the predictive model 406. The set of patient data may be the patient information 402 and the set of provider data may be the provider information 404. The set of system preference criteria may be the system preference criteria 414. In some examples, operations 604-612 may be performed by the ranking subsystem 304.

At operation 514, the patient provider matching system 130 causes display of the ranked set of search results on a graphical user interface of the first client device. Operation 514 may be performed by the UI Subsystem 308.

Software Architecture

FIG. 6 is a block diagram illustrating a software architecture 604, which can be installed on any one or more of the devices described herein. The software architecture 604 is supported by hardware such as a machine 602 that includes processors 620, memory 626, and I/O components 638. In this example, the software architecture 604 can be conceptualized as a stack of layers, where each layer provides a particular functionality. The software architecture 604 includes layers such as an operating system 612 libraries 610, frameworks 608, and applications 606. Operationally, the applications 606 invoke API calls 650 through the software stack and receive messages 652 in response to the API calls 650.

The operating system 612 manages hardware resources and provides common services. The operating system 612 includes, for example, a kernel 614, services 616, and drivers 622. The kernel 614 acts as an abstraction layer between the hardware and the other software layers. For example, the kernel 614 provides memory management, processor management (e.g., scheduling), component management, networking, and security settings, among other functionality. The services 616 can provide other common services for the other software layers. The drivers 622 are responsible for controlling or interfacing with the underlying hardware. For instance, the drivers 622 can include display drivers, camera drivers, BLUETOOTH® or BLUETOOTH® Low Energy drivers, flash memory drivers, serial communication drivers (e.g., USB drivers), WI-FI® drivers, audio drivers, power management drivers, and so forth.

The libraries 610 provide a common low-level infrastructure used by the applications 606. The libraries 610 can include system libraries 618 (e.g., C standard library) that provide functions such as memory allocation functions, string manipulation functions, mathematic functions, and the like. In addition, the libraries 610 can include API libraries 624 such as media libraries (e.g., libraries to support presentation and manipulation of various media formats such as Moving Picture Experts Group-4 (MPEG4), Advanced Video Coding (H.264 or AVC), Moving Picture Experts Group Layer-3 (MP3), Advanced Audio Coding (AAC), Adaptive Multi-Rate (AMR) audio codec, Joint Photographic Experts Group (JPEG or JPG), or Portable Network Graphics (PNG)), graphics libraries (e.g., an OpenGL framework used to render in two dimensions (2D) and three dimensions (3D) in a graphic content on a display), database libraries (e.g., SQLite to provide various relational database functions), web libraries (e.g., WebKit to provide web browsing functionality), and the like. The libraries 610 can also include a wide variety of other libraries 628 to provide many other APIs to the applications 606.

The frameworks 608 provide a common high-level infrastructure that is used by the applications 606. For example, the frameworks 608 provide various graphical user interface (GUI) functions, high-level resource management, and high-level location services. The frameworks 608 can provide a broad spectrum of other APIs that can be used by the applications 606, some of which may be specific to a particular operating system or platform.

In an example, the applications 606 may include a home application 636, a contacts application 630, a browser application 632, a book reader application 634, a location application 642, a media application 644, a messaging application 646, a game application 648, and a broad assortment of other applications such as a third-party application 640 The applications 606 programs that execute functions defined in the programs. Various programming languages can be employed to create one or more of the applications 606 structured in a variety of manners, such as object-oriented programming languages (e.g., Objective-C, Java, or C++) or procedural programming languages (e.g., C or assembly language). In a specific example, the third-party application 640 (e.g., an application developed using the ANDROID™ or IOS™ software development kit (SDK) by an entity other than the vendor of the particular platform) may be mobile software running on a mobile operating system such as IOS™, ANDROID™, WINDOWS® Phone, or another mobile operating system. In this example, the third-party application 640 can invoke the API calls 650 provided by the operating system 612 to facilitate functionality described herein.

Machine Architecture

FIG. 7 is a diagrammatic representation of the machine within which instructions 710 (e.g., software, a program, an application, an applet, an app, or other executable code) for causing the machine to perform any one or more of the methodologies discussed herein may be executed. The instructions may provide probability of provider matches. The instructions may operate for a machine learning process to develop likely provider matches. For example, the instructions 710 may cause the machine to execute any one or more of the methods described herein. The instructions 710 transform the general, non-programmed machine into a particular machine programmed to carry out the described and illustrated functions in the manner described. The machine may operate as a standalone device or may be coupled (e.g., networked) to other machines. In a networked deployment, the machine may operate in the capacity of a server machine or a client machine in a server-client network environment, or as a peer machine in a peer-to-peer (or distributed) network environment. The machine may comprise, but not be limited to, a server computer, a client computer, a personal computer (PC), a tablet computer, a laptop computer, a netbook, a set-top box (STB), a personal digital assistant (PDA), an entertainment media system, a cellular telephone, a smartphone, a mobile device, a wearable device (e.g., a smartwatch), a smart home device (e.g., a smart appliance), other smart devices, a web appliance, a network router, a network switch, a network bridge, or any machine capable of executing the instructions 710, sequentially or otherwise, that specify actions to be taken by the machine. Further, while only a single machine is illustrated, the term “machine” shall also be taken to include a collection of machines that individually or jointly execute the instructions 710 to perform any one or more of the methodologies discussed herein. The machine, for example, may comprise the member-related client device 102, member-related client device 104, or any one of a number of server devices in a patient provider matching system 130. In some examples, the machine may also comprise both client and server systems, with certain operations of a particular method or algorithm being performed on the server-side and with certain operations of the particular method or algorithm being performed on the client-side.

The machine may include processors 704, memory 706, and input/output I/O components 638, which may be configured to communicate with each other via a bus 740. In an example, the processors 704 (e.g., a Central Processing Unit (CPU), a Reduced Instruction Set Computing (RISC) Processor, a Complex Instruction Set Computing (CISC) Processor, a Graphics Processing Unit (GPU), a Digital Signal Processor (DSP), an Application Specific Integrated Circuit (ASIC), a Radio-Frequency Integrated Circuit (RFIC), another processor, or any suitable combination thereof) may include, for example, a processor 708 and a processor 708 that execute the instructions 710. The term “processor” is intended to include multi-core processors that may comprise two or more independent processors (sometimes referred to as “cores”) that may execute instructions contemporaneously. Although FIG. 7 shows multiple processors 704, the machine may include a single processor with a single-core, a single processor with multiple cores (e.g., a multi-core processor), multiple processors with a single core, multiple processors with multiples cores, or any combination thereof.

The memory 706 includes a main memory 714, a static memory 716 and a storage unit 718 both accessible to the processors 704 via the bus 740. The main memory 714, a static memory 716 and storage unit 718 both store the instructions 710 embodying any one or more of the methodologies or functions described herein. The instructions 710 may also reside, completely or partially, within the main memory 714, within the static memory 716, within machine-readable medium 720 within the storage unit 718, within at least one of the processors 704 (e.g., within the Processor's cache memory), or any suitable combination thereof, during execution thereof by the machine.

The I/O components 702 may include a wide variety of components to receive input, provide output, produce output, transmit information, exchange information, capture measurements, and so on. The specific I/O components 702 that are included in a particular machine will depend on the type of machine. For example, portable machines such as mobile phones may include a touch input device or other such input mechanisms, while a headless server machine will likely not include such a touch input device. It will be appreciated that the I/O components 702 may include many other components that are not shown in FIG. 7. In various examples, the I/O components 702 may include user output components 726 and user input components 728. The user output components 726 may include visual components (e.g., a display such as a plasma display panel (PDP), a light-emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The user input components 728 may include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or another pointing instrument), tactile input components (e.g., a physical button, a touch screen that provides location and force of touches or touch gestures, or other tactile input components), audio input components (e.g., a microphone), and the like.

In further examples, the I/O components 702 may include biometric components 730, motion components 732, environmental components 734, or position components 736, among a wide array of other components. For example, the biometric components 730 include components to detect expressions (e.g., hand expressions, facial expressions, vocal expressions, body gestures, or eye-tracking), measure biosignals (e.g., blood pressure, heart rate, body temperature, perspiration, or brain waves), identify a person (e.g., voice identification, retinal identification, facial identification, fingerprint identification, or electroencephalogram-based identification), and the like. The motion components 732 include acceleration sensor components (e.g., accelerometer), gravitation sensor components, rotation sensor components (e.g., gyroscope).

The environmental components 734 include, for example, one or cameras (with still image/photograph and video capabilities), illumination sensor components (e.g., photometer), temperature sensor components (e.g., one or more thermometers that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detection concentrations of hazardous gases for safety or to measure pollutants in the atmosphere), or other components that may provide indications, measurements, or signals corresponding to a surrounding physical environment.

The position components 736 include location sensor components (e.g., a GPS receiver component), altitude sensor components (e.g., altimeters or barometers that detect air pressure from which altitude may be derived), orientation sensor components (e.g., magnetometers), and the like.

Communication may be implemented using a wide variety of technologies. The I/O components 702 further include communication components 738 operable to couple the machine to a network 722 or devices 724 via respective coupling or connections. For example, the communication components 738 may include a network interface component or another suitable device to interface with the network 722. In further examples, the communication components 738 may include wired communication components, wireless communication components, cellular communication components, Near Field Communication (NFC) components, Bluetooth® components (e.g., Bluetooth® Low Energy), Wi-Fi® components, and other communication components to provide communication via other modalities. The devices 724 may be another machine or any of a wide variety of peripheral devices (e.g., a peripheral device coupled via a USB).

Moreover, the communication components 738 may detect identifiers or include components operable to detect identifiers. For example, the communication components 738 may include Radio Frequency Identification (RFID) tag reader components, NFC smart tag detection components, optical reader components (e.g., an optical sensor to detect one-dimensional bar codes such as Universal Product Code (UPC) bar code, multi-dimensional bar codes such as Quick Response (QR) code, Aztec code, Data Matrix, Dataglyph, MaxiCode, PDF417, Ultra Code, UCC RSS-2D bar code, and other optical codes), or acoustic detection components (e.g., microphones to identify tagged audio signals). In addition, a variety of information may be derived via the communication components 738, such as location via Internet Protocol (IP) geolocation, location via Wi-Fi® signal triangulation, location via detecting an NFC beacon signal that may indicate a particular location, and so forth.

The various memories (e.g., main memory 714, static memory 716, and memory of the processors 704) and storage unit 718 may store one or more sets of instructions and data structures (e.g., software) embodying or used by any one or more of the methodologies or functions described herein. These instructions (e.g., the instructions 710)), when executed by processors 704, cause various operations to implement the disclosed examples.

The instructions 710 may be transmitted or received over the network 722, using a transmission medium, via a network interface device (e.g., a network interface component included in the communication components 738) and using any one of several well-known transfer protocols (e.g., hypertext transfer protocol (HTTP)). Similarly, the instructions 710 may be transmitted or received using a transmission medium via a coupling (e.g., a peer-to-peer coupling) to the devices 724.

An example of provider data, which can be used to predict a provider match for a specific patient, includes the specialty of the provider. The speciality of the provider need not be the specialty based on the providers own designation, e.g., after the provider training, but can be based on the actual claims data associated with the provider over a period of time. The period of time can be a year or multiple years, a decade or the entire career of the provider.

The machine learning model to generate possible provider matches may be generated by a training engine and may be implemented in computer instructions executable by one or more processing devices of a computing device. To generate the one or more machine learning models, including the provider match machine learning model, the training engine may train the one or more machine learning models using the information collected from claims data, provider data, third party reviews, on-line posts regarding the provider, and the like. For example, the training engine may retrieve, from a database, artificial intelligence models and/or techniques, machine learning models and/or techniques, statistical models and/or techniques, other suitable models and/or techniques, or a combination thereof. The training engine may use data associated with the artificial intelligence models and/or techniques, machine learning models and/or techniques, statistical models and/or techniques, other suitable models and/or techniques, or a combination thereof to train the machine learning model. Additionally, or alternatively, the training engine may train, periodically retrain, and/or iteratively train the machine learning model using feedback provided by a user or generated by the computing device.

In some embodiments, the machine learning model may be initially trained using a supervised learning technique, an unsupervised learning technique, or a combination thereof. The machine learning model may be trained, retrained, and/or iteratively trained using at least the output of the machine learning model (e.g., using a supervised learning technique, an unsupervised learning technique, or a combination thereof).

In some embodiments, the machine learning model may include a multi-layer perceptron model or other suitable model including any suitable number of layers. For example, the machine learning model may include a fully-connected multi-layer perceptron model. The machine learning model may include input layer, a hidden layer, an output layer, other suitable layers, or a combination thereof.

The present disclosure describes methods and systems to find a medical care provider that serves a patient's unique needs. This is a highly personalized process. Embodiments herein describe a patient provider matching system for providing a personalized experience that highlights factors that are most important for patients in choosing their medical care provider. The patient provider matching system receives a query by a patient user and identifies a list of medical providers that match the query based on provider qualifications and medical claims data. The patient provider matching system, ranks the identified list of medical providers based on patient data and provider data. The patient provider matching system, displays the ranked list of medical providers on a graphical user interface for the patient user. Further details of the patient provider matching system are provided below.

What is claimed is:

1. A method comprising:
receiving a query from a patient user on a client device;
based on the query and a location of the client device, accessing medical claim data from a database;
determining a patient user location based on characteristics of the accessed medical claim data;
identifying a set of search results related to the query, the set of search results associated with the determined patient user location;
for each search result in the identified set of search results:
determining a probability that the patient user will select a provider associated with the search result based on a set of patient data associated with the patient user and a set of provider data associated with the provider, and
ranking the search result based on the determined probability and a set of system preference criteria; and
causing display of the ranked set of search results on a graphical user interface of the client device.

2. The method of claim 1, wherein determining the probability that the patient user will select the provider associated with the search result further comprises:
using a predictive model trained to analyze the set of patient data associated with the patient user and the set of provider data associated with the provider to generate the probability.

3. The method of claim 2, wherein determining the probability that the patient user will select the provider associated with the search result further comprises:
using the predictive model trained to analyze patient insights associated with the provider and review highlights associated with the provider to generate the probability.

4. The method of claim 2, wherein the predictive model is a logistics regression model.

5. The method of claim 3, wherein the review highlights associated with the provider are generated using a natural language processor trained to analyze the patient reviews associated with the provider.

6. The method of claim 1, wherein after identifying the set of search results related to the query, the method further comprises:
determining that a size of the of search results exceeds a threshold amount.

7. The method of claim 1, further comprising:
receiving a selection from the first client device, the selection corresponding to a selected result from the ranked set of search results.

8. The method of claim 1, wherein the patient user location is based on a centroid of a geographic location of the patient user.

9. The method of claim 1, wherein the patient user location is based on a centroid of a closest city to the patient user.

10. A system comprising:
a processor; and
a memory storing instructions that, when executed by the processor, configure the system to:
receive a query from a patient user on a client device;
based on the query and a location of the client device, access medical claim data from a database;
determine a patient user location based on characteristics of the accessed medical claim data;
identify a set of search results related to the query, the set of search results associated with the determined patient user location;
for each search result in the set of search results:
determine a probability that the patient user will select a provider associated with the search result based on a set of patient data associated with the patient user and a set of provider data associated with the provider, and
rank the search result based on the determined probability and a set of system preference criteria; and
cause display of the ranked set of search results on a graphical user interface of the client device.

11. The system of claim 10, wherein determining the probability that the patient user will select the provider associated with the search result further comprises:
use a predictive model trained to analyze the set of patient data associated with the patient user and the set of provider data associated with the provider to generate the probability.

12. The system of claim 11, wherein determining the probability that the patient user will select the provider associated with the search result further comprises:
use the predictive model trained to analyze patient insights associated with the provider and review highlights associated with the provider to generate the probability.

13. The system of claim 11, wherein the predictive model is a logistics regression model.

14. The system of claim 12, wherein the review highlights associated with the provider are generated use a natural language processor trained to analyze the patient reviews associated with the provider.

15. The system of claim 10, wherein after identifying the set of search results related to the query, the operations further comprises:
determine that a size of the of search results exceeds a threshold amount.

16. The system of claim 10, wherein the instructions further configure the system to:
receive a selection from the first client device, the selection corresponding to a selected result from the ranked set of search results.

17. The system of claim 10, wherein the patient user location is based on a centroid of a geographic location of the patient user.

18. The system of claim 10, wherein the patient user location is based on a centroid of a closest city to the patient user.

19. A non-transitory computer-readable storage medium, the computer-readable storage medium including instructions that when executed by a computer, cause the computer to:
receive a query from a patient user on a client device;
based on the query and a location of the client device, access medical claim data from a database;
determine a patient user location based on characteristics of the accessed medical claim data;
identify a set of search results related to the query, the set of search results associated with the determined patient user location;
for each search result in the set of search results:
determine a probability that the patient user will select a provider associated with the search result based on a set of patient data associated with the patient user and a set of provider data associated with the provider, and
rank the search result based on the determined probability and a set of system preference criteria; and
cause display of the ranked set of search results on a graphical user interface of the client device.

20. The computer-readable storage medium of claim 19, wherein determining the probability that the patient user will select the provider associated with the search result further comprises:
use a predictive model trained to analyze the set of patient data associated with the patient user and the set of provider data associated with the provider to generate the probability.
